(19) United States
(12) Patent Application Publication
(10) Pub. No.: US 2005/0113923 A1
(43) Pub. Date: May 26, 2005

(54) Prosthetic spinal disc nucleus

(76) Inventors: David Acker, St. James, NY (US); Raphael P. Davis, Port Jefferson, NY (US); Miriam H. Rafailovich, Stony Brook, NY (US)

Correspondence Address: FRISHAUF, HOLTZ, GOODMAN & CHICK, PC, 767 THIRD AVENUE, 25TH FLOOR, NEW YORK, NY, 10017-2023, US

(21) Appl. No.: 10/958,448

(22) Filed: Oct. 4, 2004

Related U.S. Application Data
(60) Provisional application No. 60/508,453, filed on Oct. 3, 2003.

(51) Int. Cl. A61F 2/44

(52) U.S. Cl. 623/17.12; 623/17.16; 623/23.72

(57) ABSTRACT

A spinal disc nucleus pulposus implant is provided which includes a biocompatible material injected into an intradiscal space in a fluid state below physiological temperatures, and cured by temperature alone via a reversible phase shift to form a gel at physiological temperatures in the intradiscal space.

### CROSS-REFERENCE TO RELATED APPLICATIONS

The present application is related to and claims the benefit of priority under 35 USC 119 of U.S. Provisional Application Ser. No.60/508,453 filed Oct. 3, 2004, the entire contents of which are incorporated herein by reference.

### FIELD OF THE INVENTION

The present invention relates to a prosthetic spinal disc nucleus.

### BACKGROUND OF THE INVENTION

When a spinal disc becomes damaged due to trauma or disease resulting in a disc herniation, it may become necessary to replace a natural spinal disc nucleus with a prosthesis. Such prostheses should preferably mimic the shape and function of the natural nucleus. Several varieties of prostheses have been proposed for replacing the natural disc nucleus.

For example, U.S. Pat. No. 5,047,055 to Bao et al discloses a prosthetic spinal disc nucleus which is made of a hydrogel material and is implanted into an intradiscal space while the implant is dehydrated. After the prosthesis is inserted, the implant is hydrated and expands to a shape conforming to the natural nucleus. In a similar invention disclosed in U.S. Pat. No. 5,192,326 also to Bao et al, the prosthetic nucleus is made of either a solid hydrogel core or plurality of hydrogel beads surrounded by a membrane. The prosthesis is implanted and hydrated to fill the intiadiscal space.

However, the prosthesis of Bao et al relies solely on the natural annulus to constrain the expanded hydrogel. This essentially uncontrolled expansion creates a lateral force that acts directly on the annulus, which is typically already damaged. Therefore, additional forces placed on the annulus by the prosthesis may impede healing and even cause further deterioration. In addition, it is difficult to accurately position such dehydrated implants within the nucleus cavity.

U.S. Pat. No. 6,602,291 to Ray et al discloses one solution to the problems encountered by the prostheses of Bao et al. Ray et al discloses a hydrogel in a constraining jacket that expands on hydration. Such a device is inserted into the intradiscal space in a first shape and hydrated after insertion to assume a second shape that fills a volume less than the volume of the intradiscal space.

The prosthesis of Ray et al, however, may still be difficult to insert. In addition, the preparation of the prosthesis outside of the patient may also create problems. And because the prosthesis of Ray et al is constructed externally, precise measurements of the implant site must be made prior to insertion.

In order to correct some of the problems encountered by externally created implants, solutions have been proposed that include a flowable material which forms the prosthesis. U.S. Pat. No. 6,443,988 to Felt et al, for example, discloses an implant which comprises a container that is inserted into the site of implantation and that is filled with a curable material, which is then cured in situ. The shape of this implant may be manipulated in situ and may also avoid problems of size and shape which would otherwise hinder implantation.

A similar prosthetic nuclear disc pulposus is disclosed in U.S. Pat. No. 6,187,048 to Milner et al. Milner et al discloses a spinal disc implant comprising acrylates which are inserted into the intradiscal space and which are induced to at least partially polymerize through the addition of a cross-linking agent. This prosthesis, however, is similar in composition to joint implants, which eventually decompose and become mobile.

Another approach to the creation of a prosthesis that hardens in situ is disclosed in U.S. Pat. No. 6,264,659 to Ross et al. The implant of Ross et al is created by heating a thermoplastic material such as gutta percha to a temperature at which it becomes flowable. The thermoplastic material is then injected into the intradiscal space and allowed to cool, thereby forming a prosthetic spinal disc nucleus.

Implants such as these, however, utilize both polymers and/or additional curing agents that must be either mixed just prior to insertion or inserted separately. Still further, these implants may not be easily reversible.

### OBJECT OF THE INVENTION

The object of the present invention is to provide a prosthetic spinal disc nucleus which closely mimics the properties of a natural spinal disc, which does not require additive curing agents, and which may be implanted through a minimally invasive procedure.

### SUMMARY OF THE INVENTION

To achieve the object of the invention, a spinal disc nucleus pulposus implant is provided which comprises a biocompatible material injected into an intradiscal space in a fluid state below physiological temperatures, and cured by temperature alone via a reversible phase shift to form a gel at physiological temperatures in the intradiscal space.

In addition, a method is provided for achieving the object of the present invention, which includes removing nucleus pulposus tissue from a spinal disc; and injecting a biocompatible material into an intradiscal space; wherein the biocompatible material is injectable into the intradiscal space in a fluid state below physiological temperatures, and is curable by temperature alone via a reversible phase shift to form a gel at physiological temperatures.

### BRIEF DESCRIPTION OF THE DRAWINGS

FIG. 1 shows the injection of the implant of the present invention;

FIG. 2 shows an injected implant;

FIG. 3 shows a cooled suction device according to the present invention;

FIG. 4 shows an alternative cooled suction device according to the present invention; and

FIG. 5 shows an implant according to the present invention comprising an outer shell and an inner shell.

### DETAILED DESCRIPTION

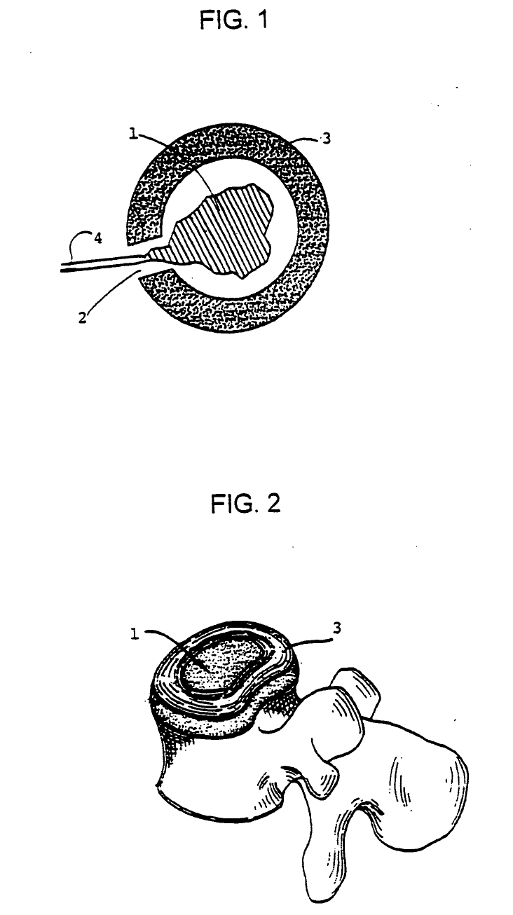

FIG. 1 shows a spinal disc nucleus pulposus implant which comprises a biocompatible material 1 that is injectable by an injection device 4, such as a needle or catheter, into an intradiscal space via a standard method of introducing a fluid into a space in the body. The material is in a fluid state below physiological temperatures, and gels at physiological temperatures. Moreover, temperature is the only necessary factor in curing the material.

The injection device 4 may, for example, comprise an angiocath.

The material is injected in a cooled fluid state through an aperture 2 in the annulus 3 of a patient that is preferably positioned in a supine position. With the patient positioned in a supine position, gravity causes the material in the cooled fluid state to flow downward and fill the intradiscal space within the annulus. In addition, by having the patient positioned in a supine position, the material in the cooled fluid state does not flow backward out of the aperture in the annulus 3 of the patient.

The material may also be implanted in the patient percutaneously via a catheter following a percutaneous discectomy. In this case, the material may be injected into the intradiscal space via the same conduit through which the percutaneous discectomy is performed.

The catheter for percutaneously injecting the material may be a microcatheter. The microcather may also include a directional port to enable control of the directionality of the flow of the material into the intradiscal space.

The aperture into which the fluid is injected is typically caused by injury but may be created artificially. The existing tissue may be emulsified by standard methods, using standard tools such as a phacoemulsifier.

The discectomy which precedes implantation of the implant of the present invention may be performed surgically, or may be performed percutaneously, as described in “Automated percutaneous discectomy, technique, indication, and clinical followup in 1000 patients,” by G. Bonaldi (Neuroradiology 45(10): 735-43, October 2003) and in “Risks and benefits of percutaneous nucleotomy for lumbar disc herniation” by J. Mochida et al (Journal of Bone and Joint Surgery (Br) 83(4): 501-05, May 2001).

Upon injection into the patient, the material in the cooled fluid state warms to physiological temperatures. As the material in the cooled fluid state warms to physiological temperatures, the material undergoes a reversible phase shift and gels and conforms to the shape of the spinal disc nucleus through natural spinal pressure exerted on the material (See FIG. 2).

Significantly, temperature is the only curing factor required for causing the injected material in the cooled fluid state to cure into a gel. The phase shift preferably occurs almost instantaneously at physiological temperatures.

In order to prevent the material from extruding out of the aperture in the annulus, the material in the cooled fluid state may be injected into the aperture in the annulus through a one way valve which prevents backflow. The one way valve may then be removed from the aperture in the annulus once the material is cured by warming.

After the material has been injected and cured by warming, the aperture in the annulus is closed by welding with RF energy or by other known tissue welding techniques.

Significantly, the implant of the present invention is “reversible” in that the cured material in the gel state at physiological temperatures may be rendered back into a fluid state by cooling, and then removed. This enables the size of the implant to be reduced, for example, by inserting a cooled suction device to remove some of the volume of the implant. The cooled suction device may be used to cool the material in the vicinity thereof so as to return the material in the vicinity of the cooled suction device to the liquid state. The cooled suction device may then be used to withdraw the material that has been returned to the liquid state to remove some of the volume of the implant.

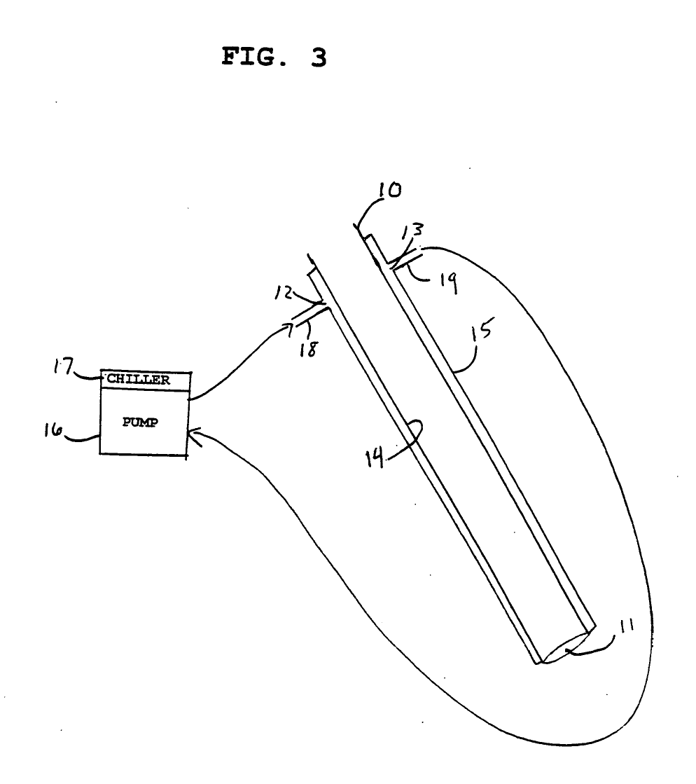

As shown in FIG. 3, the cooled suction device 10 is preferably a circulating water cooled suction device which includes a suction port 11 at the distal end thereof, a shaft 14 through which the cooled material is removed from the intradiscal space, and a cooling jacket 15 surrounding the shaft 14 and extending to the suction port 11 of the cooled suction device 10. At the proximal end of the cooled suction device, the cooling jacket 15 includes an input port 12 and an output port 13 that are connected to a pump 16 via input tube 18 and output tube 19. The pump 16 pumps chilled water, which is chilled by the chiller 17 connected to the pump 16, into the cooling jacket 15 via the input tube 18 and input port 12. The material in the vicinity of the cooled suction device 10 is thereby cooled and converted back to the fluid space for removal from the intradiscal space.

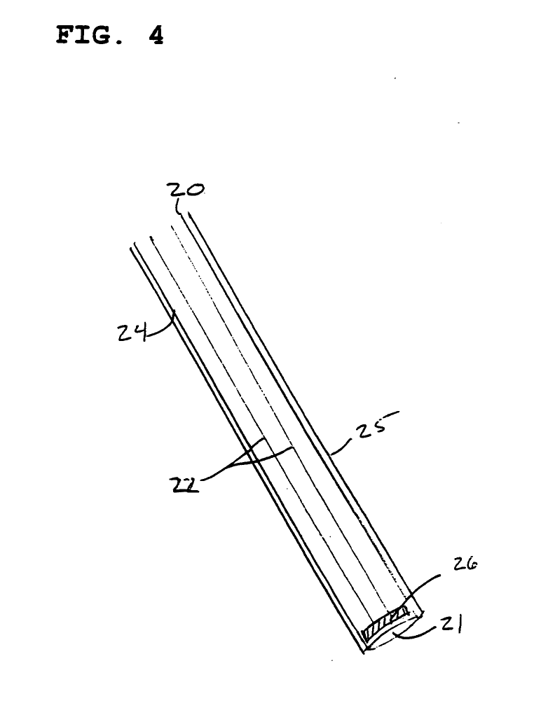

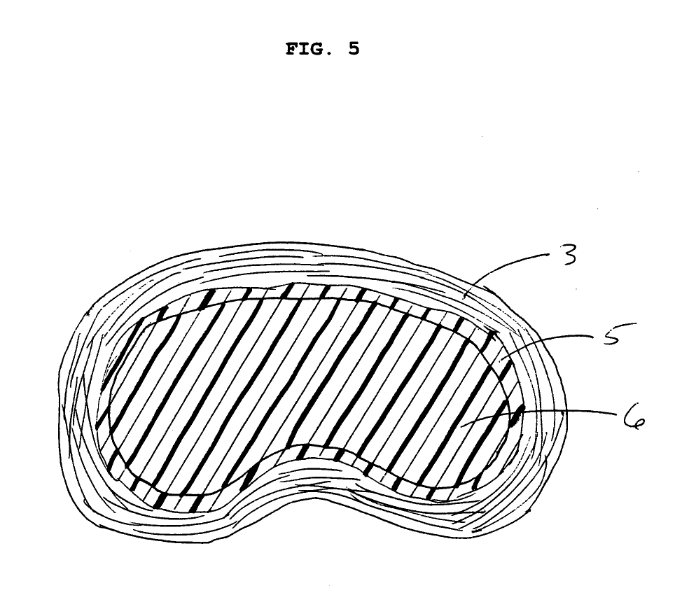

Alternatively, as shown in FIG. 4, the cooled suction device 20 may be a Peltier cooled suction device, which includes a ring Peltier cooling device 26 provided around the suction port 21 at the distal end of the cooled suction device 20. Electric leads 12 extend down the shaft 24 of the cooled suction device 20 to supply the ring Peltier cooling device 26 with electricity, and a jacket 25 covers the electric leads 12 on the shaft 24 of the cooled suction device 20. The jacket 25 may also cover the ring Peltier cooling device 26. Electricity is supplied to the ring Peltier cooling device 26 via the electric leads 12 so as to cool the ring Peltier cooling device 26 at the distal end of the cooled suction device 20. The material in the vicinity of the distal end of the cooled suction device 20 is thereby cooled and converted back to the fluid state for removal from the intradiscal space.

According to the present invention, the shafts 14 and 24 of the cooled suction devices 10 and 20 may be catheters or needles or endoscopic devices for introduction of specialized catheters. Thus, the cooled suction device of the present invention may be provided by, for example, fitting a cooling mechanism to either a small gauge catheter or to a large gauge needle. The suction may be provided by a syringe which is operated to withdraw the cooled material from the intradiscal space into the syringe. Of course, other methods and devices for cooling the implanted material may be used without departing from the scope of the present invention.

In addition, with the implant of the present invention, if the patient experiences pain or further damage to the spinal disc nucleus, additional material may be injected into the nucleus to increase the volume of the implant. Additional material may also be injected to add to the implant to compensate for degradation of the implant.

Thus, the implant of the present invention is advantageously modifiable both during and subsequent to the initial implantation procedure to allow shaping and manipulation of the size and compressibility of the implant. Significantly, the modification of the implant may be performed percutaneously. And advantageously, such modifications may be performed repeatedly.

As described hereinabove, the material that forms the implant of the present invention is injected in a cooled fluid state directly into the intradiscal space within the annulus of a patient.

As shown in FIG. 5, preferably, a material with a high viscosity is first injected into the intradiscal space to form a “shell” 5 to contain a material that has a lower viscosity 6 within the intradiscal space. The shell 5 may be created by “wetting” the interior surfaces (the annulus 3 and/or cartilaginous plate) of the intradiscal space using a catheter with a directional port to control the directionality of the flow of the material. The material is also preferably hydrophilic such that the material coats the interior surface of the intradiscal space upon injection into the space.

Once the higher viscosity material has been injected into the intradiscal space and cured into the gel state, then the material with the lower viscosity 6 is separately injected into the shell 5. According to the present invention, the material with the higher viscosity and the material with the lower viscosity are the same material.

After being injected, the higher viscosity material has a higher rigidity in the gel state, and the lower viscosity material has a lower rigidity in the gel state. With this structure of a more rigid shell 5 and a less rigid interior 6, a healthy disc nucleus is advantageously mimicked.

Use of the shell to contain the material advantageously prevents the flow of the material into locations where the material should not be present. For example, the shell prevents the material from flowing out through the rupture/incision in the annulus through with the material is inserted, or through other ruptures in the annulus.

In addition, a membrane of gelatinous foam may instead be coated on the walls of the intradiscal space so as to prevent the outflow of material before the material is cured by the physiological temperatures. The gelatinous foam may be any standard gelatinous foam used for surgical procedures.

Alternatively, a jacket or balloon may first be inserted into the intradiscal space within the annulus of the patient, with the material in the cooled fluid state being inserted into the jacket or balloon and then cured by heating to physiological temperatures within the jacket or balloon to form the implant.

The material of the implant of the present invention may, for example, comprise a pleuronic polymer such as Pluronic™ F-127, a block copolymer of ethylene oxide and propylene oxide, or a polymer with properties similar to Pluronic™ F-127 in terms of gel point and biocompatibility. Pluronic™ F-127 has an average molecular weight of 12,600 and a block length of (PEO)99-(PPO)69-(PEO)99. In particular, Lutrol F127-NF-M by BSAF may be used as the polymer.

With this material, the rigidity of the implant may be controlled by, for example, modifying the water to polymer ratio to control the viscosity of the material. At higher viscosities, the material has a higher packed micelle density to cause higher rigidity of the implanted material. The higher viscosity form of the material may thus be injected first to form the shell described above. A lower viscosity material, with a correspondingly lower packed micelle density may be injected to fill the shell, as described above.

Preferably, according to the present invention, a concentration of 25% by weight of the polymer water solution is used as the lower viscosity form of the material, while a concentration of 35% by weight of the polymer water solution is used as the higher viscosity form of the material.

In addition, the Pluronic polymer of the present invention is advantageously almost instantly cured at physiological temperatures. The free flow of the material within the patient is thereby minimized. And since the material of the implant of the present invention is biocompatible and does not require any additive curing agent, even if some of the material flows into the bloodstream, the patient is not endangered.

For example, at the concentration of 25% by weight of the polymer water solution, the F-127 and water solution has a gelation temperature below 15 degrees C. and gels after less than one minute at 15 degrees C.

Since this composition of the material of the present invention gels quickly at room temperature, a cooled syringe or other cooled/cooling device may, for example, be used to inject the material into the intradiscal space. For example, an injection device similar to the cooled suction device 10 may be operated to implant the material as well as to remove the material.

At the concentration of 25% by weight of the polymer water solution, the F-127 and water solution has a storage modulus of 20 kPa at physiological temperatures (37 degrees C.), and a frequency of 1 Hz. Since the storage modulus of the human nucleus pulposus varies within a large range of from about 1 kPa to about 30 kPa, the material of the present invention must be manipulated to accord with the load frequency, water content and age associated with the patient.

For example, the rigidity of the implant increases with increasing length and molecular weight of the polymer. However, the gelation temperature decreases with increased length and molecular weight of the polymer. The rigidity of the implant may also be increased by increasing the amount of polyethylene oxide with respect to polypropylene oxide in the polymer. Thus, for example, Pluronic™ F-68, which has a lower molecular weight and shorter block length than F-127, may be used to achieve a lower modulus than is achievable with F-127.

The strength and biocompatibility of the material may preferably be augmented by substituting a cell culture medium such as DMEM for water in the polymer solution. In addition, the rigidity and strength of the material may be increased by incorporating a low concentration of nano size clay, in the amount of 5% of the polymer concentration for example, in the solution. This addition to the solution may improve the strength of the material by 50-100%. Incorporation of the nano composite clay may also slightly increase the gelation temperature of the material.

The nano size clay may, for example, be Cloisite® 10A a natural montmorillonite modified with a quaternary ammonium salt, by Southern Clay Products, Inc. Cloisite® 6A by Southern Clay Products, Inc., may also be used as the clay.

The material of the implant of the present invention is preferably combined with a radio opaque imaging contrast agent such as iodine so as to enable the material to be visualized fluoroscopically, both during and after the implantation procedure. Any undesired flow of the material from the intradiscal space may thus be visualized.

Alternative suitable image contrast agents for radiographic imaging may include iron, calcium and barium. In addition, a suitable MRI contrast agent such as gadolinium may be combined with the biocompatible material to render the material visible via MRI. Still further, other suitable imaging contrast agents may also be combined with the biocompatible material to allow the implanted material to be seen via various imaging techniques.

In summary, the implant of material of the present invention is particularly advantageous because temperature is the only necessary curing factor so that no additional curing agents or free polymer fractions are required for curing which may enter the bloodstream.

In addition, the implant of the present invention is also particularly advantageous because the volume of the implant may easily be adjusted both upward by injecting additional material and downward by cooling and removing material by suction. As a result, the compressibility of the implant may be adjusted in response to changes in the status of the patient.

Still further, the implant of the present invention enables replacement of decaying material by cooling and removing such decaying material and thereafter injecting additional new material.

Yet still further, the material of the present invention is not limited to use in the prosthetic spinal disc nucleus. The material of the present invention may also be used to fill gaps or cavities throughout the body. For example, the material of the claimed present invention may be used to fill cranial defects, sinus cavities, gaps in bone caused by cutting during surgical procedures, or other internal spaces in the body.

The invention in its broader aspects is not limited to the specific details, representative devices, and illustrated examples shown and described herein, and various modifications may be made without departing from the spirit or scope of the general inventive concept as defined by the appended claims and their equivalents.

1. A spinal disc nucleus pulposus implant comprising:
a biocompatible material injected into an intradiscal space in a fluid state below physiological temperatures, and cured by temperature alone via a reversible phase shift to form a gel at physiological temperatures in the intradiscal space.

2. The spinal disc nucleus pulposus implant of claim 1, wherein the biocompatible material comprises Pluronic™ F-127 or a polymer having similar biocompatibility and gel point properties as Pluronic™ F-127.

3. The spinal disc nucleus pulposus implant of claim 1, wherein the biocompatible material is convertible from the gel back into the fluid state by cooling, and wherein the biocompatible material in the fluid state is removable from the intradiscal space by suction.

4. The spinal disc nucleus pulposus implant of claim 1, further comprising a casing that is insertable into the intradiscal space and into which the biocompatible material is injectable.

5. The spinal disc nucleus pulposus implant of claim 1, wherein the casing comprises a jacket or balloon that is insertable into the intradiscal space before injection of the biocompatible material.

6. The spinal disc nucleus pulposus implant of claim 1, wherein the biocompatible material comprises:
a first composition of the biocompatible material that is injected into the intradiscal space so as to form a shell defining a space within the intradiscal space; and
a second composition of the biocompatible material that is injected into the space defined by the first biocompatible material;
wherein the first composition has a higher viscosity than the second composition so that the implant is externally more rigid and internally less rigid.

7. The spinal disc nucleus pulposus implant of claim 6, wherein the first composition is applied to an interior surface of the intradiscal space to form the shell.

8. The spinal disc nucleus pulposus implant of claim 1, wherein a membrane of gelatinous foam is applied to an interior surface of the intradiscal space before injection of the biocompatible material so as to inhibit outflow of the biocompatible material from the intradiscal space.

9. The spinal disc nucleus pulposus implant of claim 1, wherein the biocompatible material is marked with an imaging contrast agent so as to enable monitoring of outflow of the biocompatible material from the intradiscal space.

10. The spinal disc nucleus pulposus implant of claim 8, wherein the imaging contrast agent is a radio opaque contrast agent.

11. A material for filling an internal space in a patient, comprising:
a biocompatible material injected into the internal space in a fluid state below physiological temperatures, and cured by temperature alone via a reversible phase shift to form a gel at physiological temperatures in the internal space.

12. A method for implanting a spinal disc nucleus pulposus implant, comprising:
removing nucleus pulposus tissue from a spinal disc; and
injecting a biocompatible material into an intradiscal space;
wherein the biocompatible material is injectable into the intradiscal space in a fluid state below physiological temperatures, and is curable by temperature alone via a reversible phase shift to form a gel at physiological temperatures.

13. The method according to claim 12, wherein the nucleus pulposus tissue is removed percutaneously via a conduit, and the biocompatible material is injected through the conduit.

14. The method according to claim 12, wherein injection of the biocompatible material comprises:
injecting a first composition of the biocompatible material into the intradiscal space so as to form a shell defining a space within the intradiscal space; and
injecting a second composition of the biocompatible material into the space defined by the first biocompatible material;
wherein the first composition has a higher viscosity than the second composition so that the implant is externally more rigid and internally less rigid.

15. The method according to claim 12, further comprising applying a gelatinous foam to an interior surface of the intradiscal space before injection of the biocompatible material so as to inhibit outflow of the biocompatible material from the intradiscal space.

16. The method according to claim 12, wherein the biocompatible material is marked with an imaging contrast agent and injection of the biocompatible is monitored via an imaging system so that any outflow of the biocompatible material from the intradiscal space is detectable.

17. The method according to claim 16, wherein the imaging contrast agent is a radio opaque contrast agent and injection of the biocompatible material is carried out under fluoroscopic observation.

18. The method according to claim 12, further comprising:
cooling at least a portion of the cured biocompatible material so as to convert the cooled portion back into the fluid state; and
removing the cooled biocompatible material in the fluid state from the intradiscal space by suction.

19. The method according to claim 12, further comprising adjusting a compressibility of the implant in accordance with changes in a status of a patient by at least one of (i) cooling at least a portion of the cured biocompatible material so as to convert the cooled portion back into the fluid state, and removing the cooled biocompatible material in the fluid state from the intradiscal space by suction, and (ii) injecting additional biocompatible material into the intradiscal space.

20. The method according to claim 12, further comprising replacing decayed biocompatible material by cooling and removing the decayed biocompatible material and then injecting additional new biocompatible material.
